(19) United States
(12) Patent Application Publication
(10) Pub. No.: US 2026/0352790 A1
(43) Pub. Date: Nov. 5, 2026

(54) Apparatus with image detector and image projector

(71) Applicant: Stichting Radboud universitair medisch centrum, Nijmegen (NL)

(72) Inventor: Stefan Laurentius Maria Hummelink, Nijmegen (NL)

(73) Assignee: Stichting Radboud universitair medisch centrum, Nijmegen (NL)

(21) Appl. No.: 19/488,601

(22) PCT Filed: May 29, 2024
(86) PCT No.: PCT/EP2024/064806

§ 371 Date: Nov. 27, 2025

(30) Foreign Application Priority Data
Jun. 1, 2023 (EP) 23176824.3

(51) Int. Cl. H04N 9/31; A61B 90/00

(52) U.S. Cl. CPC H04N 9/3185; A61B 90/36; A61B 2090/366

(57) ABSTRACT

A projection apparatus (102) for image projection of images on a subject (114). The apparatus (102) comprises an image detector (104) having a detector field of view, wherein the image detector (104) is configured to detect an input image of the subject (114). A processor (204) configured to generate an output image based on the input image, wherein the output image comprises content representing at least part of a content of the input image. An image projector (106) having a projector field of view, wherein the projector field of view overlaps the detector field of view, and wherein the image projector (106) is configured to project the output image on a target surface (208) of the subject (114).

### FIELD OF THE INVENTION

The present invention relates to image projection, more particularly to an image projection using a single field of view for both image detection and image projection. In particular the invention relates to a projection apparatus for image projection of images on a subject. The invention further relates to a method of generating a projection image.

### BACKGROUND

For surgical procedures, it is important to localize features, which may be visible on e.g. medical images, in the patient's body.

Application WO 2016/127173 A1 discloses an optical imaging system utilizes a three-dimensional (3D) light scanner to capture topography information, color reflectance information, and fluorescence information of a target object being imaged, such as a surgical patient. The system also utilizes the topography information of the target object to perform an image mapping process to project the captured fluorescence or other intraoperative images back onto the target object with enhanced definition or sharpness.

The prior art document has the disadvantage of having to use the topography information of the target to calibrate the intraoperative images before projecting them back onto the target.

Patent U.S. Pat. No. 9,510,914 B2 discloses surgical guidance and image registration are provided, in which three-dimensional image data associated with an object or patient is registered to topological image data obtained using a surface topology imaging device.

Patent U.S. Pat. No. 9,646,423 B1 discloses a method for providing augmented reality in minimally invasive surgery includes capturing pre-operative image data of internal organs of a patient, capturing intra-operative image data of the internal organs with an endoscope during a surgical procedure, registering the pre-operative image data and the intra-operative data in real time during the surgical procedure, tracking the position and orientation of the endoscope during the surgical procedure, and augmenting the intra-operative image data captured by the endoscope in real time with a rendering of at least a portion of an internal organ of the patient that is in registration with the real time intra-operative image data from the endoscope but outside of the field of view of the endoscope.

Patent U.S. Ser. No. 11/113,825 B2 discloses a projected image item tracking system that analyzes projected camera images to determine items taken from, placed on, or moved on a shelf or other area in an autonomous store.

Patent Application WO 2015/135985 A1 discloses a projection system for image projection of features on a subject. Intraoperative position markers (10) are disposed on a first body surface (6), and a moveable first detector (12) with a detection line of sight is provided.

The prior art document has the disadvantage of requiring position markers on the subject. Additionally, the prior art may involve calibrating a projected image with a detected image, which may be cumbersome and/or reduce accuracy and/or quality of the projected image.

### SUMMARY OF THE INVENTION

The present invention seeks to provide an improved image projection system for projecting one or more image representations of features of interest on a body surface of a subject. Traditional image projectors may be based on calibration to ensure the projected image representation is projected at the same location on the body surface as the location where the body surface image was captured or detected. The process of calibration may lead to a projected image that has reduced accuracy and/or quality. Also a calibration step may be inconvenient.

A known image projection apparatus may have problems to align a projected image of an image projector with a detected image of an image detector. This alignment may be difficult to perform perfectly. This may involve an image transformation which could reduce accuracy and/or quality of the projected image and may consume a relatively large processing capacity.

According to an aspect of the present invention, a projection apparatus for image projection of images on a subject comprises: an image detector having a detector field of view, wherein the image detector is configured to generate an input image of the subject; an image projector having a projector field of view, wherein the projector field of view overlaps the detector field of view, and wherein the image projector is configured to project an output image based on the input image on a target surface of the subject, wherein the output image aligns with the input image.

Because the detector field of view and the projector field of view are configured to overlap, the projector can project the image at the same position where it was detected. In this way the target of the detector may be projected in a visual representation of the detected image to indicate where it physically lies below the surface of the subject. The detector field of view and the projector field of view may overlap so that the projector field of view precisely overlays the detector field of view such that the projected image is projected to the position that was detected.

Because the projected image is projected from (exactly or approximately) the location of the detector, any deformations caused by the curvature of the surface may be avoided or strongly reduced, thereby reducing or making obsolete any image transformations to correct for such deformations.

The image detector and the image projector may be fixed relative to each other in a single device. For example the image projector and the image detector may be contained in a single housing. This may help to streamline or avoid calibration procedures. Because the image detector and the image projector are fixed relative to each other, the distance and relative orientation between them are known and static. And because the image detector and the image projector are housed in the same device, the distance between the image detector and image projector is relatively small compared to the distance from the subject to the device. This way, spatial corrections to the projected image can be avoided or reduced to a minimum.

The image detector may comprise a first optical path within the projection apparatus, the first optical path being associated with the detector field of view; the image projector may comprise a second optical path within the projection apparatus, the second optical path being associated with the projector field of view; a distance from the first optical path within the projection apparatus to the second optical path within the projection apparatus may be at most 20 millimeters, preferably at most 10 millimeters, more preferably at most 5 millimeters, even more preferably at most 1 millimeter; and the image projector may be configured to project on the target surface wherein a distance of the image projector to the target surface is at least 1 meter, preferably at least 2 meters. By providing the projector very close to the detector, the projected image does not need to be transformed very much to correct for any spatial deformations that would otherwise occur. For example, the distal parts of the optical paths within the projection apparatus, where the optical paths have their optical interface with the outside of the apparatus, may be substantially parallel to each other and be close to each other as indicated.

The image detector and the image projector may share at least part of an optical path within the projection apparatus. This way, the spatial transformations to correctly align the projected image with the detected image could be omitted, because there are no deformations. A correction for difference in image resolution or other image properties between the detection image and projection image may still be useful. For example, the distal part of the optical paths within the projection apparatus, where the optical path has its optical interface with the outside of the apparatus, may be shared by the projector and detector.

The apparatus may comprise a first optical element configured to guide an incoming light beam from the subject through the optical path and toward an image sensor of the image detector; and a second optical element configured to guide an outgoing light beam from a light source of the image projector through the optical path and toward the target surface. This provides an effective manner to realize the shared optical path.

The first optical element may comprise the second optical element and may include a reflective side and a transmissive side. This provides an effective manner to realize the shared optical path.

The reflective side may be configured to guide an incoming signal toward the image sensor and the transmissive side may be configured to receive and allow to pass a light signal from the light source of the image projector. This provides an effective manner to realize the shared optical path.

The apparatus may comprise a lens, wherein the shared optical path passes through the lens to the target surface. The shared lens may define a center of the projected and detected rays. The lens may be configured as an interface between the optical path within the apparatus and outside the apparatus.

The apparatus may comprise any number of image detectors having a further field of view of the subject, wherein the further image detectors are configured to generate further detected image of the subject. The processor may be configured to generated the output image based on multiple input images. The further image detector may be used for a distance measurement, for example. Alternatively, the further image detector may generate an input image with content that can be merged with the detection image of other image detectors to generate an output image and projected back onto the projection surface. The features of the further image detector, in particular its distance and orientation and positioning with respect to the projector and the subject, may be similar to the features described in respect of the image detector set forth. Alternatively, a further image detector may be positioned further away from the projector.

The projection apparatus may comprise an emitter configured to emit radiation at a predefined frequency toward the target surface. This can be used to illuminate the subject or to excite certain elements in the field of view, for example.

The emitter may be configured to excite the radiation with a radiation frequency corresponding to an excitation frequency of an element inside the subject, wherein the image detector is configured to detect radiation of a frequency corresponding to excitation light of said element. This is useful to detect and project radiation generated by an element inside the subject back onto the surface of the subject.

The image detector may comprise at least one of a: hyper- or multispectral camera, an infrared camera, a thermal camera, or the image detector of a laser doppler imaging system. This may allow useful images to be detected and projected back onto the subject.

The processor may be configured to map a pixel of the output image to at least one pixel of the input image, wherein an inclination of an output ray associated with the pixel of the output image corresponds to an inclination of an input ray associated with the at least one pixel of the input image, said inclinations with respect to the projection apparatus; and determine a value of the output pixel based on at least one value of the at least one input pixel mapped thereto. This provides a suitable mapping of the detection field of view with the projection field of view in terms of pixels of images being detected and/or projected.

The projector field of view may be wider or narrower than the detector field of view. Thus, the fields of view do not need to be identical.

The image detector and the image projector may have overlapping optical paths to reduce or eliminate the adjustments when mapping the detected image to the projected image. The optical paths may travel through a lens of the image projection apparatus. The lens may be configured to generate a detector field of view and a projector field of view.

Image projection apparatus may include an optical element configured to align the image detector optical path and the image projector optical path. For example, if the image detector is configured to detect the target surface and both optical paths are aligned, no adjustments are necessary when mapping the detected image pixels to the projected image pixels.

The optical element may include a reflective side and a transmissive side. The reflective side may reflect light beams emitting from the subject to the image detector. The transmissive side may allow light beams emitting from the image projector to travel through the optical element to the subject. It should be noted that the image projection apparatus may include separate reflective and transmissive optical elements instead of a combined optical element.

Image projection apparatus may include one or more image detectors configured to take one or more detected images. Each detected image may represent different features, such as different wavelengths. The projected image may be generated based on one or more detected images to generate a visual representation of the one or more features.

The image projection apparatus may also include a light emitter configured to emit radiation onto the field of view of the detector. This may be helpful for imaging techniques including, but not limited to, fluorescent dyes, laser speckle contrast imaging (LSCI), and laser doppler imaging.

Another aspect of the invention provides a method of projecting an image, the method comprising: detecting an input image of the subject, by an image detector having a detector field of view; generating, by a processor, an output image based on the input image, wherein the output image comprises content representing at least part of a content of the input image; projecting, by an image projector, the output image on a target surface of the subject, by an image projector having a projector field of view, wherein the projector field of view overlaps the detector field of view.

Another aspect of the invention provides a method of generating a projection image, the method comprising: determining a first distance between an image detector and a target surface; determining a second distance between an image projector and the target surface; determining an angle between the image projector and the image detector, wherein a vertex of the angle is at the target surface; mapping a detected pixel of a detected image to a projected pixel of the projection image based on the first distance, the second distance, and the angle so that the projected pixel represents the same position of the target surface as the detected pixel.

The person skilled in the art will understand that the features described above may be combined in any way deemed useful. Moreover, modifications and variations described in respect of the apparatus may likewise be applied to the method, and modifications and variations described in respect of the method may likewise be applied to the apparatus.

### BRIEF DESCRIPTION OF THE DRAWINGS

The present invention will be explained in further detail hereinafter based on a number of exemplary embodiments with reference to the drawings. The drawings are diagrammatic and may not be drawn to scale. Throughout the drawings, similar items may be marked with the same reference numerals. In the drawings:

FIG. 1 shows an image projection apparatus according to the present disclosure;

FIG. 2 shows an exemplary image detector and image projector according to the present disclosure:

FIG. 3 shows a sketch of an exemplary detection of a thermal image and the corresponding projected image according to the present disclosure;

FIG. 4 shows an exemplary image projection apparatus including an optical element between the image detector and the image projector according to the present disclosure;

FIG. 5 shows a first exemplary configuration of a projection apparatus including two image detectors according to the present disclosure;

FIG. 6 shows a first exemplary configuration of a projection apparatus including two image detectors according to the present disclosure;

FIG. 7 shows a first exemplary configuration of a projection apparatus including two image detectors according to the present disclosure;

FIG. 8 shows an exemplary projection apparatus configured to detect an image of a subcutaneous feature according to the present disclosure;

FIG. 9 shows another exemplary configuration of the projection apparatus according to the present disclosure;

FIG. 10 shows a sketch of an exemplary projected image, including one or more data of interest, on a surface of a subject according to the present disclosure;

FIG. 11 shows an exemplary image projected on an arm of a subject;

FIG. 12 shows an exemplary image projected on a hand of a subject;

FIG. 13 shows an exemplary image of an excited dye projected on the surface of a subject.

### DETAILED DESCRIPTION

In medicine, various difficulties arise in regard to identifying patient information. For example, identifying and localizing subcutaneous structures; and locating internal organs; patient vital signs such as temperature, blood pressure, heart rate, etc.; blood flow; burns; infections; rheumatism; cancerous cells; microorganisms; and more. For example, during surgery, it may be advantageous to identify part of a patient's vascular system before beginning surgery.

The image projection apparatus disclosed herein is in general designed to project an image on the outside surface of a subject. Thus, the image projection apparatus can be located at a reasonable working distance from the subject, for example up to 3 meters. Moreover, the image projection apparatus may have fixation means to fixate the apparatus to e.g. a wall or a ceiling or a stand, at least during operation. Alternatively or additionally, the apparatus may have a handle that allows to hold the apparatus in a hand during operation. Such fixation means or handle may be connected to the detector and projector (including the optical elements along the optical path) by means of a rigid connection and/or articulated connection.

The image projection apparatus may include a laser projector configured to focus the projected image on a remote target surface regardless of the distance between the laser projector and the target surface.

Alternatively, the image projection apparatus may include a lens system to focus the projected image on the target surface. Such projection apparatus may for example be comprise light emitting diodes (LED) and/or a liquid crystal display (LCD). The image projection apparatus may include an autofocus feature or a manual focus feature to focus the projected image on the target surface. The image projection apparatus may include sensors configured to measure the distance between the LED projector and the target surface. This distance may be fed back to the image projection apparatus to determine the alignment of the projected image.

In addition or alternatively the lens system may be configured to focus the image detector, so that the detected image is in focus. In either case the focus lens may be optimized to focus to a distance from the projection apparatus of at least 1 meter and/or at most 3 meters.

It is noted that the term detector field of view may denote herein the area or volume of the subject that the image detector is capable of detecting. It is further noted that the image detector may be capable of detecting radiation that is emitted or reflected below the surface of the subject, or signals that may originate from below the surface of the subject. For example, detecting a vascular structure below the surface of the subject. It is noted that the term projector field of view may denote herein the area of the surface of the subject onto which the projector may project the projection image. It is further noted that the detector field of view and the projector field of view may overlap. The field of view may have a conical shape with a center (the apex) at the detector/projector.

It is further noted that the surface of the subject (e.g. the skin of a patient) may not be typically a flat surface and may comprise a three dimensional profile (e.g. it may be curved and may have upstanding walls, recesses etc.) following body and organ curvatures and the like. The image detector may generate a two dimensional (2D) image of the structures within the field of view. The projected image may be a 2D image projected on the surface within the field of view. Because the projected image is projected from (exactly or approximately) the location of the detector, any deformations caused by the curvature of the surface may be avoided or strongly reduced, thereby reducing or making obsolete any image transformations to correct for such deformations.

Alternatively, the projected 2D image may be corrected for the 3D surface on which it is projected. The image projection apparatus may include a 3D sensor such as a time-of-flight camera, stereoscopic camera, etc. to adapt the projected image to the 3D profile of the surface of the subject.

The image projection apparatus may also include a light emitter configured to emit radiation onto the field of view of the detector. This may be helpful for imaging techniques including, but not limited to, fluorescent dyes, laser speckle contrast imaging (LSCI), and laser doppler imaging. For example, certain bacteria or tissue may emit light at a certain frequency when exposed to light from the light emitter. The emitted light is sensed to detect an anatomical feature of interest. In another example, the apparatus may be configured to perform laser speckle contrast imaging (LSCI) with a laser that emits light. The detector measures the minimal reflective differences of a speckly pattern of a detected image. LSCI may be useful for detecting movement of e.g. red blood cells in tissue of a subject. Additionally, the emitted light may excite certain bacteria or tissues of interest when exposed to the emitted light. The emitted light may be configured for a certain frequency to excite the object of interest. The detector may be configured to detect light the object of interest reflects when exposed to the emitted light.

If the image detector is detecting an image of a feature below the target surface, the distance between the target surface and the feature may be optionally determined. A mapping algorithm may use these distances to map the detected image to the projected image. The projected image may be generated to align with the detected image. For example, the apparatus may be configured to project a visual representation of a detected internal organ on the target surface to show where the organ is located under the surface.

There may be a margin of error for the projected image. For example, the projected image may not be perfectly aligned with the detected image. The allowable margin of error may depend on the type of image detector. For example, an image detector configured to detect a vascular structure beneath the surface of the subject may allow for a smaller margin of error as compared to a thermal image detector configured to measure temperature of the subject.

The projected image may align with an underlying structure detected by the image detector. For example, an internal organ or cancerous tissue. When projecting the projected image on the surface, a visual representation of the detected image (showing the underlying structure) is projected on the target surface. This allows medical personnel to see information in real-time directly on the patient, without having to use separate display screens. The image projection apparatus can project where the features of interest are on the surface of the subject to aid in surgeries.

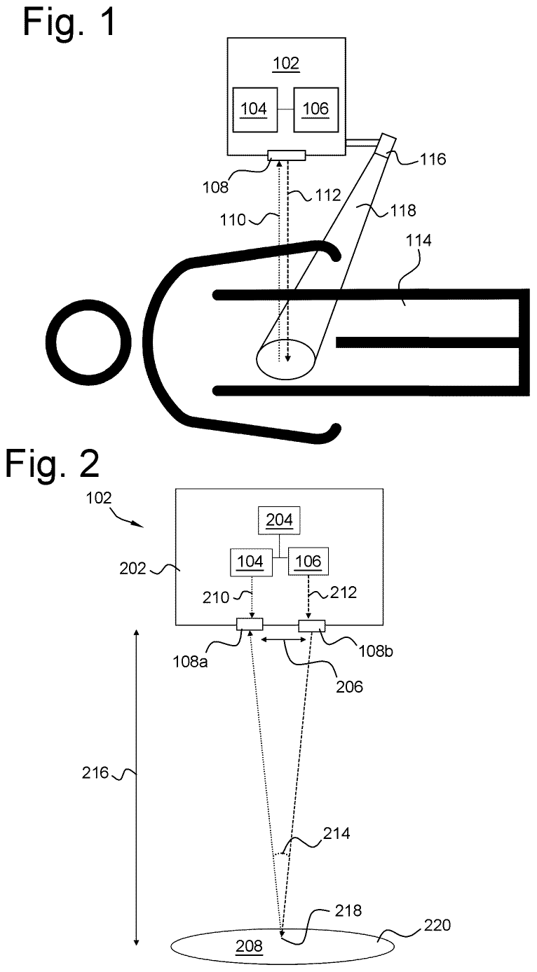

FIG. 1 illustrates an image projection apparatus 102 according to the present disclosure. Subject 114 is not part of the image projection apparatus 102. Image projection apparatus 102 may include an image detector 104 and an image projector 106 described in further detail below. The image detector 104 may be configured to detect features, such as anatomical features (organs etc.) and/or physiological features. Image projection apparatus 102 may further include a lens 108. Detected light beams 110 and projected light beams 112 may pass through lens 108 to and from image projection apparatus 102 respectively. Lens 108 may be configured to generate a field of view for image detector 104 and image projector 106. For example, the image detector field of view and the projector field of view may overlap. Lens 108 may be configured so that the projector field of view and the detector field of view overlay one another such that each pixel of the projected image is projected to exactly the same position as the corresponding pixel of the detected image. It is noted that there may be an acceptable margin of error between the projected image and the detected image. The acceptable margin of error may depend on the application and for example the type of detected image. For example, in certain applications, an image representation of body temperature of subject 114 may have a greater margin of error than an image representation of blood vessels of subject 114. The image projection apparatus may be configured for a margin of error of up to 1 cm. Said differently, the projected image could be up to 1 cm off of the object of interest's actual location. Image detector 104 may generate a detected image based on detected light beams 110 from the image detector field of view. The detected light beams may be emitted or reflected by subject 114. Image projection apparatus 102 may generate a projected image based on the detected image. Image projection apparatus 102 may map the information from the detected image to a visual representation of the information to the projected image. Image projector 106 may project the projected image based on the detected image through light beams 112. Image projector 106 may use light beams 112 to project the projected image on subject 114.

The image detector 104 may comprise a sensor, for example a sensor with a plurality of detection elements arranged in a matrix on a substrate. Each detection element may correspond to a pixel of the input image. An example sensor is a complementary metal-oxide-semiconductor (CMOS) detector. Appropriate filters may be provided so that the detector is configured to sense radiation is a specific frequency range. The image projector 106 may comprise of a laser projector or a plurality of light generating elements, such as LEDs, arranged in a matrix on a substrate. The sensors may be any camera based sensor system. Additionally, the sensors may be 2D or 3D ultrasonic devices, or an X-ray device.

For example, image projector 106 may be a laser projector configured to project the projected image using coherent parallel beams.

As another example, if image projector 106 may be an LED projector, where each light generating element may correspond to a pixel of the output image.

Although the image sensor may be configured to detect radiation in any suitable frequency range to capture any desired features, the projector may be configured to emit light in the visible light spectra, so that the image that is projected on the subject surface is visible to the human eye.

Image detector 104 and image projector 106 may have a fixed substantial parallel position within image projection apparatus 102. Image projection apparatus 102 may use the distance and angle between image detector 104 and image projector 106 to generate the projection image. Because the relative position between image detector 104 and image projector 106 is fixed, the distance and angle between them is also fixed. A single calibration is sufficient to align the optical paths and adjust the projected image over the captured sensor data onto the subject

If the paths are optically combined using an optical element as previously described and aligned in all directions, the image projection apparatus 102 uses these static values to generate the projection image without extra calculations or calibration as shown in with respect to at least FIG. 4 below.

If the paths of image detector 104 and image projector 106 are parallel to each other, the image projection apparatus 102 may use the static values to shift the projected image based on the distance between detector 104 and projector 106.

Image projection apparatus 102 may include more than one image detector 104. When image projection apparatus 102 includes multiple image detectors, each image detector may generate detected images. The projected image may be based on the plurality of detected images. For example, if image projection apparatus 102 includes a thermal image detector and an infrared image detector, e.g. filtered for Indocyanine Green (ICG), image projector 106 may project an image that represents a surface temperature of the subject 114 and indicates a presence of the dye in subject 114.

Alternatively, image detector 104 may be a Laser Doppler Imaging (LDI) system. The LDI system may include a laser exciter, a laser, and a camera configured to detect a change over time. The LDI system may be configured to detect the movement of red blood cells by reflecting a specific wavelength to determine movement of red blood cells in the tissue in the subject. For example, when the laser light reaches the tissue, the moving blood cells generate doppler components in the reflected light.

For example, LSCI may be configured to measure a burn depth in a subject. Speckles may be produced by detecting when coherent light is reflected from the tissue of the subject. The red blood cells may scatter the coherent light to cause a speckle patter to blur. Red blood cell movement may be determined based on the degree of the blur in the detected image. The burn depth can be determined based on the presence of red blood cell movement.

Typically, the surface of subject 114 is a skin surface. However, the surface of subject 114 may alternatively be an internal body surface of a subject in certain applications of image projection apparatus 102. For example, after surgery has begun and subject 114 has an open cavity, the image may be projected from outside the subject on a surface within that cavity.

For example, Image detection apparatus 102 may include radiation source 116 configured to emit a radiation such as light 118. Radiation source 116 may shine light 118 onto subject 114 to e.g. excite bacteria or e.g. excite an injected dye in or on subject 114. The excited bacteria or injected day may emit excited light when excited by light 118. Image detector 104 may be configured to detect the excited light. Radiation source 116 may also be included in an LDI system.

Alternatively, image projector 106 may also be the radiation source 116. For example, image projector 106 may include, next to the default RGB channels, an infrared channel to project an infrared light to excite an injected dye. By alternating image projector 106 between projecting from the infrared channel and the RGB channels, image projector 106 may project a stable projected image and also excite e.g. the injected dye.

This is accomplished by alternating between the infrared channel and the RGB channels, e.g., every 1/50^{th} of a second. Image projection apparatus 102 may also include image detector 104 which synchronized with the infrared channel of image projector 106 to capture the excited light every e.g. 1/50^{th} of a second. The human eye can only detect at approximately 50 frames per second (Hz), and because the image projector is capable of projecting at higher frequencies, the projected image remains stable to the human eye even though image projector 106 is alternating between projections from the RGB channel and the infrared channel.

Image detector 104 may be able to detect the excited dye faster than 1/50^{th} of a second and synchronized with the cycle of image projector 106 to project infrared light to detect the light emitted by an excited dye. Image detector 104 may be configured to detect multiple wavelengths. While image projector 106 and image detector 104 are synchronized and capable of projecting and detecting fast enough to maintain a stable projected image, image projector 106 may project two or more wavelengths in between RGB projections. Below is a table to illustrate the alternating projections and detections:

| Image Projector 106 | Image Detector 104 |
|---|---|
| Project Image (RGB) | |
| Project Wavelength 1 | Detect Wavelength 1 |
| Project Wavelength 2 | Detect Wavelength 2 |
| Project Image (RGB) | |
| Project Wavelength 1 | Detect Wavelength 1 |
| Project Wavelength 2 | Detect Wavelength 2 |

Image detection apparatus 102 may include a processor (not illustrated) configured to generate the projected image based on the detected image. Image detection apparatus 102 may use the processor to map a pixel from the detected image to the projected image. Each pixel of the detected image may be transformed to a pixel of the projected image. Each transformed pixel of the projected image may be a visual representation of the information image detector 104 detects.

Image detection apparatus 102 may be configured with static measurements of the relative orientation (angle) and distance between image detector 104 and image projector 106. The image detection apparatus 102 may be configured to map a pixel of the detected image to a pixel of the projected image based on the relative orientation and distance. The resulting projected image then considers and corrects for any difference in position between image detector 104 and image projector 106. Because the difference in position is fixed, a mapping algorithm may generate the projected image without having to obtain measurements. As a result, generating the projected image requires less computations and less time as compared to generating an image that requires obtaining measurements such as a distance between the image detector and the subject surface. It will be understood that the distance and orientation actually relates to the distal part of an optical path of the image detector 104, which ends at the lens 108 and a distal part of an optical path of the image projector 106, which also ends at the lens 108. That is, not taking into account any internal mirrors etc. In particular, the distance between the centers (apex) of the projection field of view and the detection field of view may be static. It is observed that in certain embodiments, the optical path of the image detector 104 has a different lens than the optical path of the image projector.

Image detection apparatus 102 may further include a mounting device. The mounting device may be configured to statically mount image detection apparatus 102 to a wall or ceiling. Additionally, the mounting device may include a ball and joint connector to allow image detection apparatus 102 to point in different directions. It should be noted that other types of connector may be used. Furthermore, the mounting device may include an extension to move image detection apparatus 102 towards or away from a target subject. It should be noted that any combination of mounts and extenders may be included in image detection apparatus 102. Image detection apparatus 102 may have 6 degrees of freedom to point image detection apparatus 102 in any angle and at different distances from the target subject.

Alternatively, image detection apparatus 102 may include a handle to hold image detection apparatus 102. A user may rigidly hold image detection apparatus 102 using the handle to easily point image detection apparatus 102 in any direction the user desires even without an extension arm or connector.

FIG. 2 illustrates an exemplary image projection apparatus 102 which includes a device with a housing 202. The device may include image detector 104 with lens 108a and image projector 106 with lens 108b. Image detector 104 and image projector 106 may be coupled to processor 204. Processor 204 may be configured to execute an algorithm for mapping pixels of the projected image to pixels of the detected image, and calculating the value of a pixel of the projected image based on the pixel(s) of the detected image mapped thereto.

Because image detector 104 and image projector 106 are fixed within a single device, the distance 206 between image detector 104 and image projector 106 is known and static. In particular the distance between the lens 108a of the image detector 104 and the lens 108b of the image projector is static. The entire optical paths inside the device, illustrated by means of beams 210 and 212, may be statically arranged within the device. By virtue of this fixed and known arrangement, the output image can be calculated from the input image using only the distance 216 between the device/apparatus 102 and the surface 208 of the subject surface 220 as an additional measurement. In certain embodiments, a default value for the distance 216 may be assumed in the algorithms, so that a measurement of the distance 216 may be omitted. This distance 216 may also be used for focusing of the projected images on the surface 208.

For example, the distance 206 between the image detector 104 and image projector 106, in particular between their lenses 206 and optical paths inside the housing 202 of the apparatus 102, may be at most 20 millimeters, preferably at most 10 millimeters, more preferably at most 5 millimeters, even more preferably at most 1 millimeter, even more preferably they coincide. These limits on the distance 206 may provide sufficient accuracy when using the device 102 at distances 216 from the subject surface 208 that are common in e.g. surgery. For example, the distance 216 may be in a range of 1-3 meter. For example, the focal distance of the projector is capable to focus for projecting on a surface somewhere in a range of 1-3 meters from the device 102. Such a small distance helps to make the projection accurate, even when the apparatus 102 is movable. The distance may be large enough to provide space for e.g. surgery or other treatment. Alternatively, limits on the distance 206 may be expressed relative to the distance 214 to the subject surface. Assuming a minimal working distance 216 of 0.25 meters, the relative distance 206 between the detector and the projector relative to the distance between the device 102 and the subject surface 208 may be at most 2%, preferable at most 1%, more preferably at most 0.5%, even more preferably at most 0.1%.

The lens 108a and 108b has been illustrated as a single lens for ease of discussion. However, each illustrated lens may comprise a plurality of serially arranged lenses.

Image projection apparatus 102 may include one or more sensors fixed to the housing 202 to determine the distance 216 between the (lenses of the) image projection apparatus or device and the subject surface 208. For example, the image projection apparatus may include an ultrasonic sensor or optical sensor to determine distance 216. Processor 204 may calculate an angle 214 between image detector 104 and image projector 106 based on distance 206 and 216 where the angle vertex 218 is on the subject surface 208.

Image detector 104 and image projector 106 may have the same field of view of target surface 220. Processor 204 may map the detected image of image detector 104 to the projected image of image projector 106. Processor 204 may execute a mapping algorithm to generate the projected image. The mapping algorithm may receive angle 214 and the detected image. The mapping algorithm may generate the projected image based on the detected image and angle 214 so that a visual representation of the detected image is displayed on the subject surface 208. Image projector 106 projects the projected image in field of view of target surface 220 that image detector 104 uses to detect the detected image. Because the distance 206 between (the lenses 108a, 108b of) image detector 104 and image projector 106 is constant and distances 210 and 212 and angle 214 may be quickly determined, the mapping algorithm may be able to quickly and accurately generate the projected image as compared to calibrations of image detectors and image projectors housed separately.

This may result in less processing resources to implement image projection apparatus 102. Additionally, the reduced complexity of generating the output image may result in less deformation, and therefore better quality, output images. Any manual configurations are further simplified.

Processor 204 may be configured to execute the mapping algorithm so that the projected image displays the information on subject surface 208 in the same position where image detector 104 detected it. The mapping algorithm may allow for a margin of error and the margin of error may depend on the type of image detection. In other words, the projected image may not be in exactly the same position on subject surface 208 as the detected image.

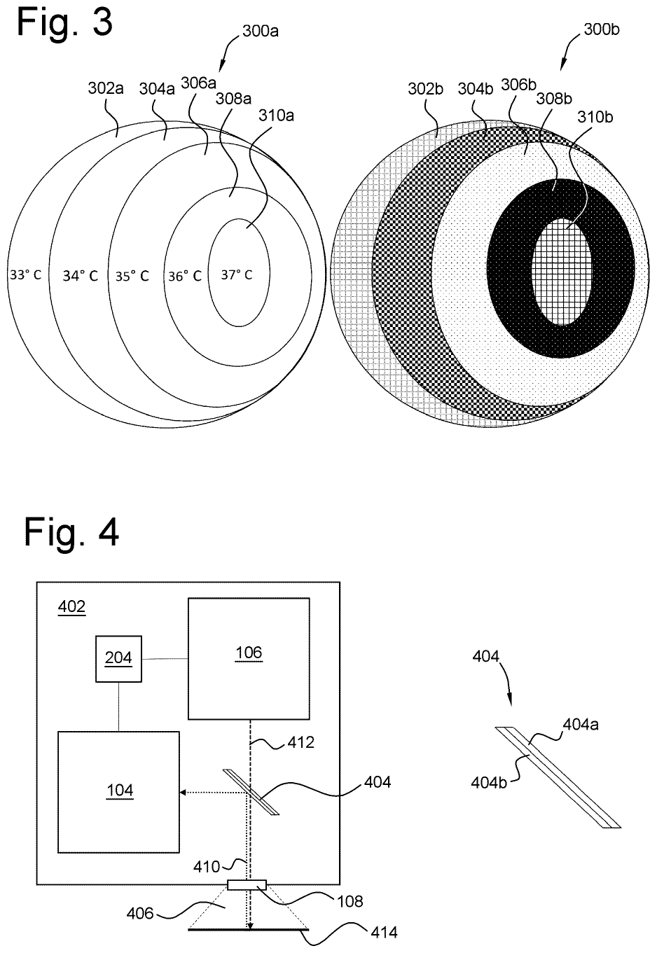

FIG. 3 illustrates a detected image 300a and a projected image 300b for thermal image data on the surface of a subject. Detected image 300a may represent field of view of target surface 220. Image detector 104 may be a thermal image camera configured to detected the temperature of the subject surface. Detected image 300a may detect that area 302a is 33 °C, area 304a is 34 °C, area 306a is 35 °C, area 308a is 36 °C, and area 310a is 37 °C. The mapping algorithm may generate projected image 300b which is a visual representation of detected image 300a. For example, projected image 300b may include 5 areas to represent the different temperatures of detected image 300a. Each area 302b-310b may correspond to areas 302a-310b. Each area 302b may have a different color to quickly represent the temperature without having to read. For example area 310b may be red to represent the warmest temperature. Area 308b may be orange, area 306b may be yellow, area 304b may be green, and area 302b may be blue to illustrate the degrading temperatures. In the drawing, these colors have been illustrated by means of gray scales.

Image projector 106 may be configured to project projected image 300b on the same position of subject surface 208 as detected image 300a was to illustrate the temperature of the subject surface 208 directly on that subject surface 208.

FIG. 4 illustrates an exemplary image projection apparatus 402 including an optical element 404. Optical element 404 includes transmissive side 404a configured to allow light beams 412 to travel through optical element 404. Optical element 404 may also include reflective side 404b configured to reflect light beams 410. It should be noted that transmissive side 404a and reflective side 404b may be separate optical elements.

Optical element 404 may be in a position between lens 108, image detector 104, and image projector 106. Optical element 404 may be configured so that image detector 104 and image projector 106 have the same field of view 406. Light beams 410 emanating or originating from subject surface 414 may reach the lens 108, which guides the light beams 410 towards reflective side 404b of optical element 404. Reflective side 404b may reflect light beams 410 to image detector 104 so that image detector 104 can detect an image of subject surface 414 from field of view 406. Projector 106 emits beams of light towards and through the transmissive side 404a of the optical element 404, and through the lens 108 towards the subject surface 414. Because of the optical element 404, image projector 106 may project the projected image from exactly the same position where image detector 104 detected the image, thus providing a mapping between input image pixels and output image pixels, which mapping is not dependent on a distance between the apparatus 402 (or lens 108) and the subject surface. Because image projector 106 is configured to project from the same position as image detector 104 detects, processor 204 may execute a second mapping algorithm without the need for determining the distance between image detector 104 and subject surface 414. Similarly, processor 204 may execute the second mapping algorithm without the need for determining the distance between image projector 106 and subject surface 414.

For example the second mapping algorithm may take as input the detected image and generate a projected image based on the detected image without adjusting the mapping of the pixels based on a distance. However, in certain embodiments, if image detector 104 is configured to detect a feature below the subject surface 208, sensors may determine the distance between the subject surface and the feature. A mapping algorithm may use this distance to generate the projected image.

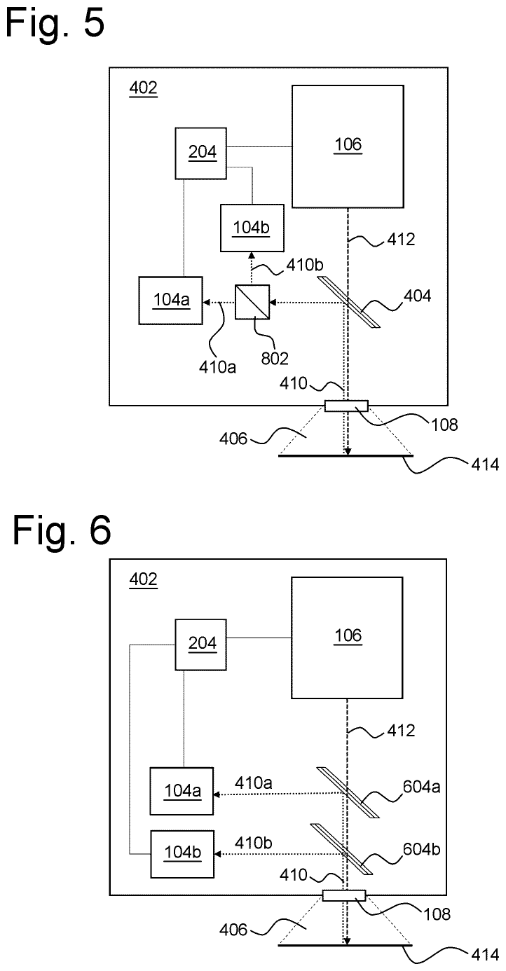

FIG. 5 illustrates image projection apparatus 402. Image projection apparatus may include image detector 104a and image detector 104b. Image detectors 104a and 104b and image projector 106 have the same field of view. Both image detectors 104a and 104b may be configured to detect an image of the subject surface 414. Beamsplitter 802 may be configured to receive and split light beams 410 between image detectors 104a and 104b. Beamsplitter 802 may be configured to split light beams 410 equally between image detectors 104a and 104b. Alternatively, beamsplitter 802 may be configured to divide light beams 410 unequally between image detectors 104a and 104b. Image detectors 104a and 104b may be configured to generate detected images based on split light beams 410a and 410b respectively. Image detectors may be configured to generate different detected images. It should be noted image projection apparatus 402 may include any number of beamsplitters to accommodate any number of image detectors. Processor 204 may be coupled to image detectors 104a and 104b and configured to generate an output image based on a combination of input images from all image detectors.

FIG. 6 illustrates image projection apparatus 402 including image detector 104a and image detector 104b. Both image detectors 104a and 104b may be configured to detect an image of the subject surface 414. Image projection apparatus 402 may include optical elements 604a and 604b configured with a reflective side and a transmissive side as described for optical element 404 with respect to FIG. 4. Optical element 604b may be in a position between lens 108 and optical element 604a to receive light beam 410 before reaching optical element 404a. Optical element 404b may be configured to only reflect a specific wavelength of light beam 410 to image detector 104b. Reflected light beam 410b may be a single wavelength or a range of wavelengths. The remaining wavelengths of light beam 410 pass through optical element 604b and reach optical element 604a. Optical element 604a may be configured to reflect a specific wavelength or range of wavelengths to image detector 410a. Alternatively, it may be configured to reflect all wavelengths. It should be noted that any number of optical elements can used to reflect specific wavelengths to any number of image detectors configured to detect the corresponding wavelengths. For example, optical element 604b may be configured to reflect infrared wavelengths to image detector 104b configured for detecting infrared light and optical element 604a may be configured to reflect visible wavelengths to image detector 104a configured for detecting visible light. The detected images from image detectors 104a and 104b may be combined to generate a visible projected image including representations of the detected infrared light and visible light.

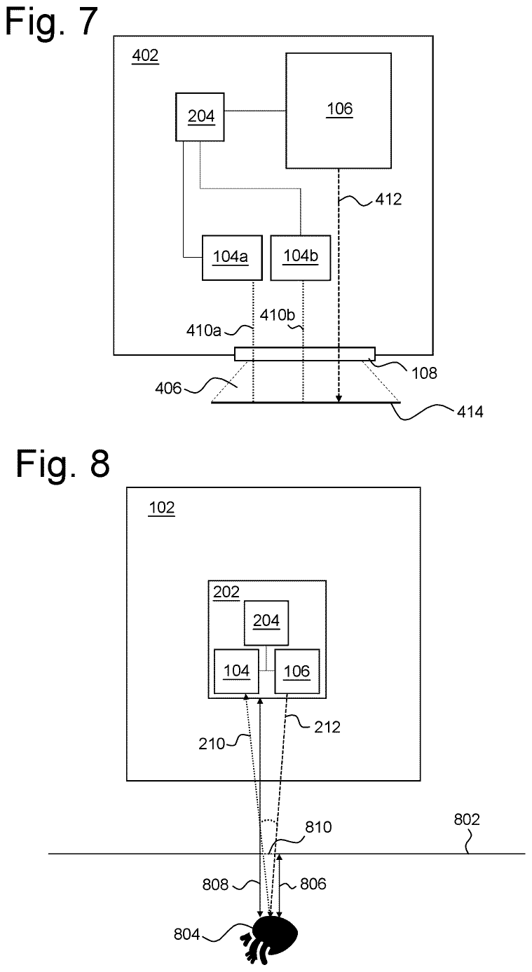

FIG. 7 illustrates image projection apparatus 402 including image detector 104a and image detector 104b. Image detectors 104a and 104b may be configured to detect different features. The respective optical paths of image detector 104a, image detector 104b, and image projector 106 may be configured in parallel. Image detectors 104a and 104b may have a fixed distance and/or orientation relative to each other. The detected images of 104a and 104b may be corrected based on the horizontal and/or vertical distance to be combined. For example, a detected image of image detector 104a may be shifted based on the horizontal and/or vertical distance relative to image detector 104b to align with a detected image of image detector 104b. The combined image may be further shifted based on a horizontal and/or vertical distance between image detector 104b and image projector 106 to generate the projected image.

FIG. 8 illustrates image projection apparatus 102 configured to including image detector 104 and image projector 106. Although, optical element 404 is not illustrated in FIG. 8, optical element 404 may be included. Image detector 104 may be configured to detect feature 804 below subject surface 802. Image projection apparatus 102 may be configured to determine the distance 808 between image detection apparatus and feature 804. Image projection apparatus 102 may be configured to determine the distance 806 between target surface 802 and feature 804. Alternatively, image projection apparatus may be configured to determine different distances, such as the distance between the feature 804 and image detector 104, image projector 106 and target surface 802, or whatever distances are necessary to generate the projected image. A mapping algorithm may use the distance 808 and 806 and any other determined distances to generate projected image so that the projected image aligns with underlying feature 804. For example, distance 810 (represented by the dotted line on target surface 802) between the light beams 210 and 212 at target surface 802. A mapping algorithm may also use distance 810 to shift a position of pixels when generating the projected image so that the projected image aligns with underlying feature 804.

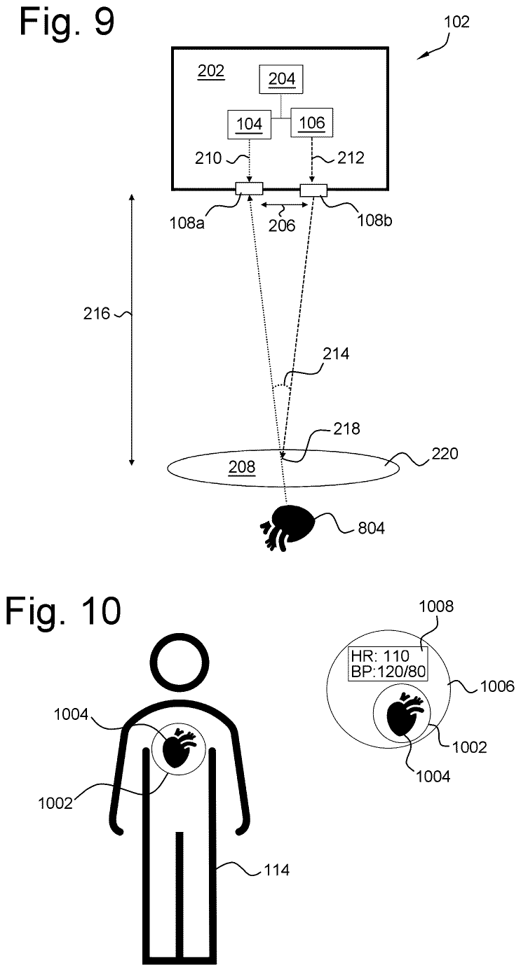

FIG. 9 illustrates an image projection apparatus configured for a case where the distance to a subcutaneous structure is not available. Since the distance to the subject surface can be measured, an incoming beam (i.e., a pixel of the input image), can be mapped to a point 218 on the subject surface 220, where the incoming beam intersects the subject surface 220. Via this point 218, the angle 214 may be determined and/or a corresponding pixel on the output image can be calculated by the processor. This way a proper mapping with negligible error may be realized, without needing any depth information of the detection image.

FIG. 10 illustrates projecting projected image 1002 on subject 114. Projected image 1002 may be a visual representation of a detected image and includes visual representation 1004 of detected feature 804. Projected image 1002 may visually represent underlying feature 804 of the target surface of subject 114 to illustrate where feature 804 is located below the target surface. Alternatively, in another example, projected image 1006 may include more than a visual representation of the detected image. Projected image 1006 includes projected image 1002, which is a visual representation of the detected image. Additionally, projected image 1006 may also include data 1008 and project the data on the target surface. The data 1008 may be projected on the target surface in a position that does not interfere with the projection of feature of interest 804.

The data 1008 may be included on the projected image 1006 in a configuration where the projector field of view is larger than the detector field of view. This allows image projector 106 to project data 1008 to subject surface without interfering with the visual representation of feature 1004. Alternatively, the detector field of view can be larger, but only a portion of the detector field of view may be projected back by the projector.

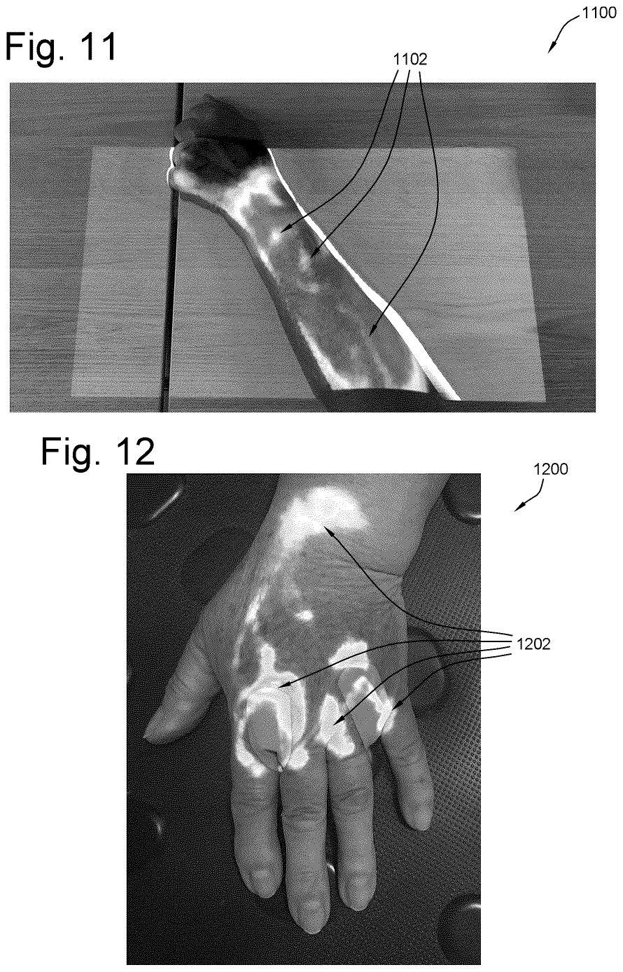

FIG. 11 illustrates an image 1100 of an arm of a subject, for example subject 114. An image of a visual representation of thermal data is projected back on the surface of the arm as shown in image 1100. Image 100 is the result of cooling the arm of the subject. A gelpack stored at 4 degrees Celsius positioned on the surface the subject's arm for 30 seconds, cooled the surface of the arm. An image projection apparatus configured to detect and project a visual representation of thermal data on the subject. The image projection apparatus included a thermal camera configured to detect thermal data of the subject's arm. The image projection apparatus was positioned approximately 50 centimeters above the table where the subject's arm laid. As the arm of the subject warmed up, the thermal camera detected the areas of the arm that warmed up first, or hotspots 1102. As the surface of subject's arm warmed up, the hotspots 1102 revealed various blood vessels below the skin's surface to show the blood vessel pattern in image 1100. The hand, was not cooled down and is represented by white or light coloring in image 1100. The arm that was cooled down is represented by the dark or black coloring in image 1100. The image projection apparatus also included a laser projector to project image the visual representation of thermal data on the surface of the subject's arm. Throughout the dark arm, a pattern of lighter coloring 1102 emerges as the arm warms revealing the pattern of blood vessels as shown in image 1100.

FIG. 12 illustrates an image 1200 of a hand of a subject, for example subject 114. The subject was injected with an Indocyanine Green (ICG) dye to reveal the lymph system in the subject's hand. The ICG was injected in two webspaces of the hand. Bandages were placed over the injection locations, as shown in image 1200. The ICG dye spread through the subject's lymph system. An image projection apparatus configured to detect and project a visual representation of the ICG dye generated image 1200. The image projection apparatus included a an infrared LED as radiation emitter 116 configured to excite the ICG injected into the subject's hand with light such as light 118. The LED may be configured for different intensities to detect more or less fluorescent light. The image projection apparatus also included a camera configured to detect the fluorescent light emitted from the excited ICG dye. The camera may be configured for different image contrast, brightness, or other image settings. The image projection apparatus is positioned approximately 30 centimeters above the subject's hand. The camera detects the fluorescent light emitting from the excited ICG. The image projection apparatus includes a projector to project an image of a visual representation of the fluorescent light on the surface of the subject's hand. Throughout the hand a green light, represented by light 1202, reveals the fluorescent light emitted by the excited ICG in the lymph system in the hand of the subject.

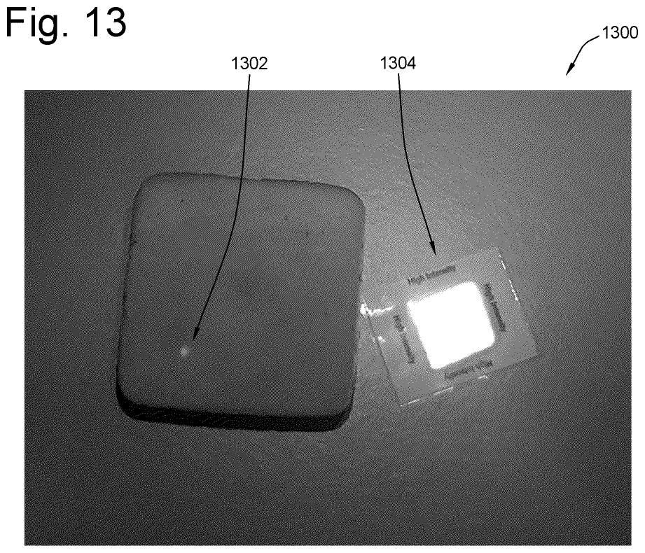

FIG. 13. Illustrates an image 1300 of an experiment with ICG dye. ICG dye was injected into an experimental pad at point 1302. A material 1304 was soaked in ICG dye. An image projection apparatus configured to detect and project a visual representation of the ICG dye generated image 1300. The image projection apparatus, similar to that of FIG. 12, is placed 30 centimeters above experimental pad and material 1304. Image 1300 shows the ICG dye at point 1302 of the experimental pad and the ICG dye in soaked material1304. The image projection apparatus is includes an LED configured to excite the ICG dye and a camera configured to detect the light emitting from the excited ICG dye. The image projection apparatus also includes a projector configured to project an visual representation of the detected fluorescent light emitting form the excited ICG dye on the surface of the subject as shown in image 1300. For example, the experimental pad and the soaked material.

Some or all aspects of the invention may be suitable for being implemented in form of software, in particular a computer program product. This applies for example to the software of the control unit 204 and the software to control the image detector and projector, and the software to process the detected image and convert it into the projected image. The computer program product may comprise a computer program stored on a non-transitory computer-readable media. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be executable by one or more processors.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

Modifications and alternative implementations of some parts or elements are therefore possible, and are included in the scope of protection as defined in the appended claims.

Certain examples are disclosed in the following clauses.
1. A projection apparatus for image projection of images on a subject comprising:
an image detector having a detector field of view, wherein the image detector is configured to detect an input image of the subject;
a processor configured to generate an output image based on the input image, wherein the output image comprises content representing at least part of a content of the input image;
an image projector having a projector field of view, wherein the projector field of view overlaps the detector field of view, and wherein the image projector is configured to project the output image on a target surface of the subject, wherein the output image aligns with the input image.
2. The projection apparatus of clause 1, wherein the image detector and the image projector are fixed relative to each other in a single device.
3. The projection apparatus of any preceding clause, wherein
the image detector comprises a first optical path within the projection apparatus, the first optical path being associated with the detector field of view;
the image projector comprises a second optical path within the projection apparatus, the second optical path being associated with the projector field of view;
a distance from the first optical path within the projection apparatus to the second optical path within the projection apparatus is at most 20 millimeters, preferably at most 10 millimeters, more preferably at most 5 millimeters, even more preferably at most 1 millimeter; and
the image projector is configured to project on the target surface wherein a distance of the image projector to the target surface is at least 1 meter, preferably at least 2 meters.
4. The projection apparatus of any preceding clause, wherein the image detector and the image projector share at least part of an optical path within the projection apparatus.
5. The projection apparatus of clause 4, further comprising
a first optical element configured to guide an incoming light beam from the subject through the optical path and toward an image sensor of the image detector; and
a second optical element configured to guide an outgoing light beam from a light source of the image projector through the optical path and toward the target surface.
6. The projection apparatus of clause 5, wherein the first optical element comprises the second optical element and includes a reflective side and a transmissive side.
7. The projection apparatus of clause 6, wherein the reflective side is configured to guide an incoming signal toward the image sensor and the transmissive side is configured to receive and allow to pass a light signal from the light source of the image projector.
8. The projection apparatus of any one of clauses 4 to 7, further comprising a lens, wherein the shared optical path passes through the lens to the target surface.
9. The projection apparatus of any one of preceding clauses,
further comprising a second image detector having a second detector field of view of the subject, wherein the second image detector is configured to generate a second input image of the subject, and
wherein the processor is configured to generate the output image further based on the second input image.
10. The projection apparatus of any one of the preceding clauses, further comprising an emitter configured to emit radiation at a predefined frequency toward the target surface.
11. The projection apparatus of clause 10, wherein the emitter is configured to excite the radiation with a radiation frequency corresponding to an excitation frequency of an element inside the subject, wherein the image detector is configured to detect excitation light of said element.
12. The projection apparatus of any one of the preceding clauses, wherein the image detector comprises at least one of a: hyper- or multispectral camera, an infrared camera, a thermal camera, or an image detector of a laser doppler imaging system.
13. The projection apparatus of any one of the preceding clauses, wherein the processor is configured to:
map a pixel of the output image to at least one pixel of the input image, wherein an inclination of an output ray associated with the pixel of the output image corresponds to an inclination of an input ray associated with the at least one pixel of the input image, said inclinations with respect to the projection apparatus; and
determine a value of the output pixel based on at least one value of the at least one input pixel mapped thereto.
14. The projection apparatus of any one of the preceding clauses, further comprising fixation means to fixate the apparatus during operation or a handle to hold the apparatus in a hand during operation.
15. A method of projecting an image, the method comprising:
detecting an input image of the subject, by an image detector having a detector field of view;
generating, by a processor, an output image based on the input image, wherein the output image comprises content representing at least part of a content of the input image;
projecting, by an image projector, the output image on a target surface of the subject, by an image projector having a projector field of view,
wherein the projector field of view overlaps the detector field of view.

1. A projection apparatus for image projection of images on a subject comprising:
an image detector having a detector field of view, wherein the image detector is configured to detect an input image of the subject;
a processor configured to generate an output image based on the input image, wherein the output image comprises content representing at least part of a content of the input image, wherein the processor is configured to map a pixel of the output image to at least one pixel of the input image, wherein an inclination of an output ray associated with the pixel of the output image corresponds to an inclination of an input ray associated with the at least one pixel of the input image, said inclinations with respect to the projection apparatus, and determine a value of the output pixel based on at least one value of the at least one input pixel mapped thereto;
an image projector having a projector field of view, wherein the projector field of view overlaps the detector field of view, and wherein the image projector is configured to project the output image on a target surface of the subject, wherein the output image aligns with the input image.

2. The projection apparatus of claim 1, wherein the image detector and the image projector are fixed relative to each other in a single device.

3. The projection apparatus of claim 1, wherein
the image detector comprises a first optical path within the projection apparatus, the first optical path being associated with the detector field of view;
the image projector comprises a second optical path within the projection apparatus, the second optical path being associated with the projector field of view;
a distance from the first optical path within the projection apparatus to the second optical path within the projection apparatus is at most 20 millimeters, preferably at most 10 millimeters, more preferably at most 5 millimeters, even more preferably at most 1 millimeter; and
the image projector is configured to project on the target surface wherein a distance of the image projector to the target surface is at least 1 meter, preferably at least 2 meters.

4. The projection apparatus of claim 1, wherein the image detector and the image projector share at least part of an optical path within the projection apparatus.

5. The projection apparatus of claim 4, further comprising
a first optical element configured to guide an incoming light beam from the subject through the shared at least part of the optical path and toward an image sensor of the image detector; and
a second optical element configured to guide an outgoing light beam from a light source of the image projector through the shared at least part of the optical path and toward the target surface.

6. The projection apparatus of claim 5, wherein the first optical element is the second optical element and includes a reflective side and a transmissive side.

7. The projection apparatus of claim 6, wherein the reflective side is configured to guide an incoming signal toward the image sensor and the transmissive side is configured to receive and allow to pass a light signal from the light source of the image projector.

8. The projection apparatus of claim 4, further comprising a lens, wherein the shared optical path passes through the lens to the target surface.

9. The projection apparatus of claim 1, further comprising a second image detector having a second detector field of view of the subject, wherein the second image detector is configured to generate a second input image of the subject, and
wherein the processor is configured to generate the output image further based on the second input image.

10. The projection apparatus of claim 1, further comprising an emitter configured to emit radiation at a predefined frequency toward the target surface.

11. The projection apparatus of claim 10, wherein the emitter is configured to excite the radiation with a radiation frequency corresponding to an excitation frequency of an element inside the subject, wherein the image detector is configured to detect excitation light of said element.

12. The projection apparatus of claim 1, wherein the image detector comprises at least one of a: hyper- or multispectral camera, an infrared camera, a thermal camera, or an image detector of a laser doppler imaging system.

13. The projection apparatus of claim 1, wherein the processor is configured to:
determine a first distance between an image detector and a target surface;
determine a second distance between an image projector and the target surface;
determine an angle between the image projector and the image detector, wherein a vertex of the angle is at the target surface; and
map a detected pixel of a detected image to a projected pixel of the projection image based on the first distance, the second distance, and the angle.

14. The projection apparatus of claim 1, further comprising fixation means to fixate the apparatus during operation or a handle to hold the apparatus in a hand during operation.

15. A method of projecting an image, the method comprising:
detecting an input image of the subject, by an image detector having a detector field of view;
generating, by a processor, an output image based on the input image, wherein the output image comprises content representing at least part of a content of the input image, wherein said generating comprises mapping a pixel of the output image to at least one pixel of the input image, wherein an inclination of an output ray associated with the pixel of the output image corresponds to an inclination of an input ray associated with the at least one pixel of the input image, said inclinations with respect to the projection apparatus, and determining a value of the output pixel based on at least one value of the at least one input pixel mapped thereto;
projecting, by an image projector, the output image on a target surface of the subject, by an image projector having a projector field of view,
wherein the projector field of view overlaps the detector field of view.
